(19) United States
(12) Patent Application Publication
(10) Pub. No.: US 2015/0175518 A1
(43) Pub. Date: Jun. 25, 2015

(54) METAL-ORGANIC FRAMEWORKS

(71) Applicant: The University of Nottingham, Nottingham (GB)

(72) Inventors: Martin Schroder, Nottingham (GB); Martyn Poliakoff, Nottingham (GB); Peter Bayliss, Nottingham (GB); Eduardo Velilla, Nottingham (GB)

(21) Appl. No.: 14/415,880

(22) PCT Filed: Jul. 19, 2013
(86) PCT No.: PCT/GB2013/051949

§ 371 Date: Jan. 20, 2015

(30) Foreign Application Priority Data
Jul. 20, 2012 (GB) 1212898.9

(51) Int. Cl. C07C 51/41; C07F 1/08; C07F 5/06

(52) U.S. Cl. CPC C07C 51/418; C07F 5/069; C07F 1/08

(57) ABSTRACT

The present invention relates to metal-organic frameworks and, in particular, a continuous flow process for synthesising a metal-organic framework comprising the steps of: providing a ligand and a metal salt which are suitable for forming a metal-organic framework, mixing the ligand and metal salt with a solvent to form a mixture, and providing the mixture at a temperature sufficient to cause the ligand and the metal salt to react to form a metal-organic framework. The invention also relates to a method for the treatment of a metal-organic framework to extract unreacted ligand from the metal organic framework, a method for synthesising a metal-organic framework using recycled unreacted ligand, and uses for metal-organic frameworks.

### FIELD OF THE INVENTION

The present invention relates to metal-organic frameworks and, in particular, a continuous flow process for synthesising a metal-organic framework comprising the steps of: providing a ligand and a metal salt which are suitable for forming a metal-organic framework, mixing the ligand and metal salt with a solvent to form a mixture, and providing the mixture at a temperature sufficient to cause the ligand and the metal salt to react to form a metal-organic framework. The invention also relates to a method for the treatment of a metal-organic framework to extract unreacted ligand from the metal organic framework, a method for synthesising a metal-organic framework using recycled unreacted ligand, and uses for metal-organic frameworks.

### BACKGROUND TO THE INVENTION

Metal-organic frameworks are the focus of significant scientific interest and research. They are currently one of the most promising materials for gas storage. Of particular interest is the potential to store lower carbon fuels, such as methane or hydrogen. However, for use in industrial applications metal-organic frameworks must not only possess the desired functionality but are also required to be thermally and chemically robust as well as being cost effective. In order for metal-organic frameworks to meet these demanding criteria they will also require a synthetic process that is scalable with high output at relatively low economic and environmental cost.

On the other hand due to the potential large scale of production it is highly desirable for this process to be as sustainable as possible. The aim of sustainable or green chemistry is to improve existing and develop new processes and products to eliminate the use of and generation of hazardous substances.

The most commonly used methods for metal-organic framework synthesis are solvothermal batch reaction in Teflon-lined stainless steel bombs or glass pressure tubes. Long reactions times of several days are commonly used with slow heating for prolonged periods and protracted cooling rates. They often use solvents such as dimethylformamide (DMF), which is toxic, mutagenic and environmentally hazardous. Moreover, the substantial volumes of solvent required for solvothermal syntheses mean that disposal of waste solvent is a significant concern.

There is therefore an ongoing need for metal-organic frameworks with improved properties, improved processes for manufacturing metal-organic frameworks, and improved methods of treatment for metal-organic frameworks.

The present invention addresses these and other problems with the prior art.

### SUMMARY OF THE INVENTION

Accordingly, in a first aspect, the present invention provides a continuous flow process for synthesising metal-organic frameworks. The process typically comprises the steps of: providing a ligand and a metal salt which are suitable for forming a metal-organic framework, mixing the ligand and metal salt with a solvent, preferably water and/or ethanol, to form a mixture, preferably a single, substantially homogenous mixture, and providing the mixture at a temperature sufficient to cause the ligand and the metal salt to react to form a metal-organic framework. Typically, the solvent is extracted from the pores of the metal-organic framework following synthesis. Solvent extraction may be performed using any method known in the art, including by evaporation or by the use of a supercritical fluid.

The metal-organic framework synthesised according to the invention may comprise a single species of ligand and a single species of metal salt; however, the process of the invention may equally be used to produce mixed ligand metal-organic frameworks and/or mixed metal metal-organic frameworks, and should be construed accordingly. Providing a ligand and a metal salt; therefore, also includes providing a plurality of ligands and/or a plurality of metal salts.

In a preferred embodiment, the solvent, preferably water and/or ethanol, is preheated. Preferably, the solvent, preferably water and/or ethanol, is pre-heated to a temperature above that of the ligand and the metal salt, or solutions comprising the same. Typically, when the ligand and/or metal salt are provided in a solution, the continuous medium is the same as the solvent. Preferably, the solvent, preferably water and/or ethanol, is preheated to a temperature sufficient to cause the ligand to react with the metal salt to form a metal-organic framework. Preferably, the solvent, preferably water and/or ethanol, is preheated to at or above the temperature at which the mixture is provided at in order for the ligand and metal salt to react to form a metal-organic framework. Typically, the solvent, preferably water and/or ethanol, is preheated to a temperature of at least about 150° C., preferably at least about 200° C., preferably above 250° C., preferably 300° C. In embodiments, the solvent, preferably water and/or ethanol, is supercritical or near critical solvent, preferably supercritical or near critical water and/or ethanol. It is preferable to preheat the solvent otherwise it is necessary to heat the reagents once they are mixed in the reaction vessel; thereby lowering the efficiency of the process as a whole.

A near critical fluid will typically be at a temperature substantially above the boiling point of the liquid, but below the critical temperature, and a pressure greater than the vapour pressure, whilst ensuring a single liquid phase. The pressure may be greater than, less than or at the critical pressure if the temperature is below the critical temperature. Near critical fluids may have a temperature up to about 175° C., preferably about 100° C., preferably about 50° C., below the critical temperature of the substance in question. Typically, at least one of the solvent properties of a near critical fluid will be substantially different from the value observed for that property in the ambient liquid state. Typically, the exact temperature and pressure can be selected in order to optimise ligand removal.

The critical point of water is 374° C. and 22 MPa. Near critical water may be understood to be water at a temperature of from about 200° C. to about 374° C. and at a pressure greater than about 1.55 MPa, preferably from about 1.55 MPa to MPa to about 22 MPa.

The critical point of ethanol is 241° C. and 6.3 MPa. Near critical ethanol may be understood to be ethanol at a temperature from about 150° C. to about 241° C., preferably from about 200° C. to about 241° C., and at a pressure of at least 5 MPa and preferably from about 5 MPa to about 22 MPa, preferably from about 5 MPa to about 8 MPa.

Preferably, the solvent, preferably water and/or ethanol, is at a temperature such that the organic ligand is soluble therein. Preferably, the solvent, preferably water and/or a mixture of water and ethanol, is at a temperature of from about 150° C. to about 500° C., preferably from about 200° C. to about 500° C., preferably from about 220° C. to about 375° C., from about 240° C. to about 280° C. is particularly preferred. Where ethanol alone is used, preferably, the ethanol is at a temperature of from about 100° C. to about 400° C., preferably from about 125° C. to about 300° C., from about 200° C. to about 250° C. is particularly preferred. Typically, the solvent, preferably water and/or ethanol, and/or the mixture are at a pressure of from about from 1.55 MPa to about 23 MPa.

Near critical water is particularly preferred because, without wanting to be bound by theory, it is believed that at near critical conditions, the dielectric constant of water reaches values typical of organic solvents and therefore water can solubilise organic compounds such as the ligands. It is also believed that the ionic product of water also increases reducing the need for addition of acid or base to the reaction. Water is also better for the environment than alternative organic solvents.

Ethanol is a solvent that may provide alternative solubilising conditions and/or be used with water sensitive ligands/metal organic frameworks. Ethanol is better for the environment than other organic solvents as it can be produced sustainably.

The present invention has the advantage of providing short reaction times for the synthesis of the metal-organic framework. In preferred embodiments, the reaction time is less than about one hour, preferably less than about 30 minutes, more preferably less than 10 minutes, most preferably from about 4 minutes to about 7 minutes. Without wishing to be bound by theory, it is believed that the higher temperature provides faster reaction kinetics and therefore reaction times are reduced compared to other methods for synthesising metal-organic frameworks.

In a preferred embodiment, following synthesis, the metal-organic framework is allowed to cool to room temperature, preferably using a heat exchanger.

Typically, the metal salt and ligand are provided in separate dispersions, preferably separate solutions, preferably separate aqueous and/or ethanolic solutions.

In an embodiment of the invention the ligand is provided in a solution, preferably an aqueous and/or ethanolic solution, with a concentration of from about 0.01 mol dm⁻³ to about 0.5 mol dm⁻³, more preferably about 0.10 mol dm⁻³ to about 0.25 mol dm⁻³, more preferably about 0.15 mol dm⁻³ to about 0.225 mol dm⁻³. Typically, higher ligand concentrations increase productivity; however, they also increase the risk of blockages in the continuous flow reaction vessel. Typically, the ligand, or solution thereof, is provided at a temperature of from about 0° C. to about 80° C., more preferable at a temperature of from about 10° C. to about 40° C. Typically, the ligand, or solution thereof, is provided at ambient or standard temperature and pressure.

In an embodiment of the invention the metal salt is provided in a solution, preferably an aqueous and/or ethanolic solution, with a concentration of from about 0.01 mol dm⁻³ to about 0.5 mol dm⁻³, more preferably about 0.10 mol dm⁻³ to about 0.25 mol dm⁻³, more preferably about 0.15 mol dm⁻³ to about 0.225 mol dm⁻³. Typically, increasing the metal salt solution concentration increases productivity; however, the increase in concentration also increases the risk of blockages in the continuous flow reaction vessel. Typically, the metal salt, or solution thereof, is provided at a temperature of from about 0° C. to about 80° C. to about, more preferable at a temperature of from about 10° C. to about 40° C. Typically, the metal salt, or solution thereof, is provided at ambient or standard temperature and pressure.

Appropriate flow rates for the continuous flow process would be selected by the skilled person depending on the rate at which metal-organic framework needs to be produced. For instance, laboratory and industrial processes may require different flow rates. Typically, a flow rate is selected such that a significant proportion, preferably substantially all, of the ligand and metal salt react to form a metal-organic framework. Typically, the flow rate of the ligand and metal salt is selected, for a given concentration of ligand, such that the yield of metal-organic framework is maximised whilst the residence time is as short as possible.

Typically, the concentration of the mixture comprising the ligand, the metal salt in the near critical water and/or ethanol is from about 0.003 mol dm⁻³ to about 0.16 mol dm⁻³, more preferably about 0.03 mol dm⁻³ to about 0.09 mol dm⁻³, more preferably about 0.05 mol dm⁻³ to about 0.075 mol dm⁻³.

The reaction between the ligand and the metal salt is typically conducted in a continuous flow reactor. Preferably, the reactor has a volume of from about 10 ml to about 50 ml, more preferably from about 15 ml to about 25 ml. Although, it will be appreciated by the skilled person, that the invention may equally be carried out on a laboratory or an industrial scale, and so may be conducted in reactors outside of the above preferred range.

Typically, the ligand, metal salt and near critical water and/or ethanol, are pumped into the continuous reactor.

The present invention provides an improved space time yield compared to known processes. In a preferred embodiment, the space time yield for the continuous flow process of the invention is at least about 500 kg m⁻³ d⁻¹, preferably at least about 1200 kg m⁻³ d⁻¹.

In a second aspect, the present invention provides a method for the treatment of a metal-organic framework to remove impurities and increase the available internal surface area and/or porosity. The process typically comprises the steps of:
a. providing a metal-organic framework formed by reacting a ligand and a metal salt;
b. introducing a supercritical fluid or near critical fluid into the metal-organic framework; and
c. removing the supercritical fluid or near critical fluid;
   characterised in that unreacted ligand is soluble in the supercritical fluid or near critical fluid.

In embodiments, the metal-organic framework is provided free from the solvent used in its manufacture, although, in alternative embodiments, the solvent, or traces thereof, may still be present. For the avoidance of doubt, the metal-organic framework may be provided by a method other than that described in the first aspect of the invention.

The metal-organic framework treated according to the invention may comprise a single species of ligand and a single species of metal salt; however, the process of the invention may equally be used to treat mixed ligand metal-organic frameworks and/or mixed metal metal-organic frameworks, and should be construed accordingly. Providing a metal-organic framework formed by reacting a ligand and a metal salt; therefore, also includes providing a metal-organic framework formed by reacting a plurality of ligands and/or a plurality of metal salts.

Typically, at least about 75% by weight, more preferably at least about 80% by weight, more preferably at least about 90% by weight, more preferably from about 80% to about 98% by weight, more preferably from about 90% to about 95% by weight of the unreacted ligand is removed.

Using a supercritical fluid is preferred because they are able to remove any residual solvent from the synthesis of the metal-organic framework. Furthermore, because supercritical fluids have low surface tension, there is reduced risk of causing the metal-organic framework to collapse during the process, which may occur when using other fluids, particularly liquid solvents. Also, by using a supercritical or near critical fluid in which the unreacted ligand is soluble it is possible to remove any unreacted ligand from within the metal-organic framework and thereby increase the metal-organic framework's available internal surface area and/or porosity and/or gas storage capacity. Typically, before the supercritical or near critical fluid is introduced, liquid of the same substance is pumped over the metal-organic framework. The metal-organic framework will then be exposed to supercritical or near critical conditions. In an embodiment of the invention, supercritical or near critical fluid is pumped through the metal-organic framework. Typically, the metal-organic framework is not soluble in the supercritical or near critical fluid.

Typically, the available internal surface area and/or porosity and/or gas storage capacity are significantly increased by this process. The internal surface area, porosity and gas storage capacity may be measured using a Quantachrome Autosorb-1 (model no. As1-GYTKXL11, software ver. 1.61).

Certain supercritical fluids, such as carbon dioxide, will not dissolve some unreacted ligands, and so may not suitable for use without a co-solvent in the present invention.

In a preferred embodiment, the supercritical or near critical fluid is polar, preferably organic, preferably an alcohol, more preferably ethanol; although the supercritical fluid may be selected from the group consisting of ethanol, methanol, propanol, isopropanol, butanol, acetone, chloroform, fluoroform, CF₃CH₂F, ammonia, dimethyl ether, diethyl ether.

In an embodiment of the invention, the supercritical or near critical fluid is used in combination with a co-solvent. Typically, the co-solvent is a supercritical or near critical fluid. A particularly preferred co-solvent is supercritical carbon dioxide. The combination of supercritical ethanol with a supercritical carbon dioxide cosolvent is particularly preferred. This combination has been found to be particularly useful in the removal of unreacted ligand and solvent, when the solvent used to form the metal-organic framework contained DMF.

Typically, the metal-organic framework is not soluble in the co-solvent or combined supercritical or near critical fluid and co-solvent.

Preferably the supercritical fluid or near critical fluid is at a temperature of at least about 150° C., preferably at least about 200° C., preferably at least 235° C., more preferably at least about 250° C. It will be understood that the selected temperature should not cause decomposition of the metal-organic framework.

Preferably, the supercritical or near critical fluid is at a pressure of at least 6.3 MPa, most preferably about 6.3 MPa to about 25 MPa, from about 6.3 MPa to about 15 MPa is particularly preferred.

A near critical fluid will typically be at a temperature substantially above the boiling point of the liquid, but below the critical temperature, and a pressure greater than the vapour pressure, whilst ensuring a single liquid phase. The pressure may be greater than, less than or at the critical pressure if the temperature is below the critical temperature. Near critical fluids may have a temperature up to about 175° C., preferably about 100° C., preferably about 50° C., below the critical temperature of the substance in question. Typically, at least one of the solvent properties of a near critical fluid will be substantially different from the value observed for that property in the ambient liquid state. Typically, the exact temperature and pressure can be selected in order to optimise ligand removal.

The critical point of ethanol is 241° C. and 6.3 MPa. Near critical ethanol may be understood to be ethanol at a temperature from about 150° C. to about 241° C., preferably from about 200° C. to about 241° C., and at a pressure of at least 5 MPa and preferably from about 5 MPa to about 22 MPa, preferably from about 5 MPa to about 8 MPa.

In a third aspect, the present invention provides a method for synthesising a metal-organic framework using recycled unreacted ligand comprising the steps of:
a. providing a first metal-organic framework formed by reacting a ligand and a metal salt;
b. extracting any unreacted ligand present in the first metal-organic framework; and
c. using said extracted unreacted ligand to synthesise a second metal-organic framework.

In preferred embodiments, the first and/or second organic frameworks are synthesised according to the continuous flow process according to the first aspect of the invention. The recycling process may thus form a part of a continuous flow process for the manufacture of a metal-organic framework. Recycling of unreacted reagents, particularly the ligand(s), is advantageous as these materials often represent a significant proportion of the costs associated with the production of metal organic frameworks.

The metal-organic framework synthesised according to the invention may comprise a single species of ligand and a single species of metal salt; however, the process of the invention may equally be used to produce mixed ligand metal-organic frameworks and/or mixed metal metal-organic frameworks, and should be construed accordingly. Providing a first metal-organic framework formed by reacting a ligand and a metal salt; therefore, also includes providing a first metal-organic framework formed by reacting a plurality of ligands and/or a plurality of metal salts.

In still further embodiments, the unreacted ligand is extracted according to the method of the second aspect of the invention.

In a fourth aspect, the invention provides metal-organic frameworks synthesised or treated according to the processes or methods of any of the preceding aspects of the invention.

In fifth aspect, the invention provides the use of a metal-organic framework according to the fourth aspect of the invention in gas storage, carbon capture, gas and substrate separation, sensing, drug delivery, photo-optics, magnetic devices and catalysis.

In sixth aspect, the invention provides the use of a supercritical fluid to remove unreacted ligand from a metal-organic framework. Typically, the unreacted ligand is soluble in the supercritical fluid. Preferably, the supercritical fluid is supercritical ethanol. Preferably, supercritical carbon dioxide is used as a cosolvent.

In a seventh aspect, the invention provides the use of near critical liquid or supercritical fluid, preferably water and/or ethanol, in the manufacture of a metal-organic framework using a continuous flow process. Typically, the near critical or supercritical water and/or ethanol is used as a solvent for the reaction used to form the metal-organic framework.

It will be appreciated by the skilled person, that all of the aspects and embodiments of the invention may be combined mutatis mutandis.

### DESCRIPTION OF THE FIGURES

The above-mentioned and other features and objects of this invention, and the manner of obtaining them, will become more apparent and the invention itself will be better understood by reference to the following description of embodiments of the invention taken in conjunction with the accompanying drawings, wherein:

FIG. 1 is a schematic of the continuous flow process according to the invention.

FIG. 2 is a schematic of the method for treating a metal-organic framework according to the invention.

FIG. 3 shows Powder X-ray diffraction plots.

FIG. 4 shows thermogravimetric analysis of as-synthesised product.

FIG. 5 shows a comparison of N₂ isotherms at 77K for batch and continuous flow product.

FIG. 6 shows PXRD of reaction product at different temperatures (150° C., 200° C. and 250° C.) and a simulated pattern for terephthalic acid.

FIG. 7 shows the ATR-IR spectra of as synthesised MIL-53(Al), MIL-53(Al) post scEtOH extraction, and terephthalic acid.

FIG. 8 show a TGA plot for both as synthesised and ligand removed by scEtOH samples.

FIG. 9 shows a Type I N₂ isotherm at 77 K for new MIL-53 (Al) sample treated with scEtOH.

FIG. 10 shows a powder diffraction of the sample before and after scEtOH washing, and after N₂ adsorption.

FIG. 11 is a powder diffraction that shows that after 24 hrs of storage in air after gas adsorption, the phase is almost completely hydrated.

FIG. 12 is a schematic of an alternative arrangement to that shown in FIG. 2 of the method for treating a metal-organic framework according to the invention.

FIG. 13 is a close-up of the extraction vessel in FIG. 12.

FIG. 14 shows a comparison of the PXRD of the as-synthesised samples from continuous flow synthesis at 150° C. and 200° C., to the simulated HKUST-1 pattern.

FIG. 15 shows an IR spectra of the as-synthesised HKUST-1 material produced by continuous flow synthesis at 150° C. and 200° C.

FIG. 16 shows a thermogravimetric analysis of as-synthesised HKUST-1 material from continuous flow reaction at 150° C.

FIG. 17 shows a thermogravimetric analysis (TGA) of as-synthesised HKUST-1 material from continuous flow reaction at 200° C.

FIG. 18 shows an N₂ isotherm of 200° C. CF product HKUST-1 material from the reaction at 200° C. in continuous flow.

Although the drawings represent exemplary embodiments of the present invention, the drawings are not necessarily to scale and certain features may be exaggerated to better illustrate and explain the invention. The exemplification set out herein illustrates exemplary embodiments of the invention only.

### DETAILED DESCRIPTION OF THE INVENTION

The present invention provides a continuous flow process for synthesising a metal-organic framework typically comprising the steps of: providing a ligand and a metal salt which are suitable for forming a metal-organic framework, mixing the ligand and metal salt with solvent, preferably water and/or ethanol, to form a mixture, and providing the mixture at a temperature sufficient to cause the ligand and the metal salt to react to form a metal-organic framework.

For the purpose of the invention, a continuous flow process is understood to be one in which the chemical reaction is run in a flowing stream rather than as a batch. The stream can be run continuously, although it may be stopped and restarted, if, for instance, the continuous flow reactor needs to be cleaned, replaced or otherwise maintained, or for stopped for any other reason.

Continuous flow reactors are well-known in the art. They are typically tube-like. They are typically made from materials that will not react with the reagents of the continuous flow process, for instance from stainless steel, glass and/or polymers. The reagents are pumped into the reactor and react once they mix. Mixing may be by diffusion or mechanical agitation or by the turbulent flow of the reagents. Continuous flow reactors allow good control over reaction conditions including heat transfer, residence time, and mixing.

The residence time is calculated from the volume of the reactor and the volume flow rate through the reactor. To achieve longer residence time, reagents can be pumped more slowly and/or a larger volume reactor used. In contrast, in batch production residence time is defined by how long a vessel is held at a given temperature.

Reaction stoichiometry is defined by the concentration of reagents and the ratio of their flow volume.

Metal-organic frameworks typically comprise two major components: a metal ion, or cluster of metal ions, and a ligand scaffold. The ligands are typically mono-, di-, tri-, or tetradentate. The choice of metal ion and linker has significant effects on the structure and properties of the metal-organic framework. For example, the coordination preference of the metal influences the size and shape of pores by dictating how many ligands can bind to the metal and in which orientation. Metal ions are typically provided by means of a metal salt, preferably a metal salt solution. The present invention is not limited to any specific metal salts or ions, ligands or metal organic frameworks.

The ligand may be any ligand suitable for forming a metal-organic framework. Typically, the ligand is at least a bidentate organic compound. Typically, the ligand is selected from the group consisting of organic bidentate, tridentate, tetradentate, or more generally, polydentate compounds that are capable of coordinating to metal ions. The term “at least bidentate organic compound” as used within the scope of the present invention typically refers to an organic compound comprising at least one functional group which is able to form at least two bonds, preferably two coordinative bonds, to a given metal ion and/or to form one coordinative bond each to two or more, preferably two metal atoms.

Examples of functional groups to be mentioned, via which the said coordinative bonds can be formed, include the following functional groups in particular: —CO₂H, —CS₂H, —NO₂, —B(OH)₂, —SO₃H, —Si(OH)₃, —Ge(OH)₃, —Sn(OH)₃, —Si(SH)₄, —Ge(SH)₄, —Sn(SH)₃, —PO₃H, —AsO₃H, —AsO₄H, —P(SH)₃, —As(SH)₃, —CH(RSH)₂, —C(RSH)₃, —CH(RNH₂)₂, —C(RNH₂)₃, —CH(ROH)₂, —C(ROH)₃, —CH(RCN)₂, —C(RCN)₃, where R, for example, is preferably an alkylene group having 1, 2, 3, 4 or 5 carbon atoms such as e.g. a methylene, ethylene, n-propylene, propylene, n-butylene, i-butylene, t-butylene or n-pentylene group or an aryl group containing one or two aromatic moieties such as e.g. two C₆ rings which may or may not be condensed and, independently of one another, can be substituted in a suitable manner by at least one substituent each, and/or which, independently of one another, can each contain at least one heteroatom such as e.g. N, O and/or S. In accordance with likewise preferred embodiments, functional groups should be mentioned in which the abovementioned R group is not present. To be mentioned among these are, inter alfa, —CH(SH)₂, —C(SH)₃, —CH(NH₂)₂, —C(NH₂)₃, —CH(OH)₂, —C(OH)₃, —CH(CN)₂ or —C(CN)₃.

The at least two functional groups can in principle be bound to any suitable organic compound, as long as there is the assurance that the organic compound having these functional groups is capable of forming the coordinative bond and of producing the framework material.

The organic compounds comprising the at least two functional groups are preferably derived from a saturated or unsaturated aliphatic compound or an aromatic compound or a compound which is both aliphatic and aromatic.

The aliphatic compound or the aliphatic moiety of the both aliphatic and aromatic compound can be linear and/or branched and/or cyclic, a plurality of cycles per compound also being possible. More preferably, the aliphatic compound or the aliphatic moiety of the both aliphatic and aromatic compound comprises from 1 to 15, more preferably from 1 to 14, more preferably from 1 to 13, more preferably from 1 to 12, more preferably from 1 to 11 and particularly preferably from 1 to 10 C atoms such as e.g. 1, 2, 3, 4, 5, 6, 7, 8, 9 or 10 C atoms. Particularly preferred in this context are, inter alia, methane, adamantane, acetylene, ethylene or butadiene.

The aromatic compound or the aromatic moiety of the both aromatic and aliphatic compound can have one ring or alternatively more than one ring such as e.g. 2, 3, 4 or 5 rings, with the option of the rings being separate and/or at least two rings being present in condensed form. Particularly preferably, the aromatic compound or the aromatic moiety of the both aliphatic and aromatic compound has 1, 2 or 3 rings, one or two rings being especially preferred. Independently of one another, each ring of the abovementioned compound may further comprise at least one heteroatom such as N, O and/or S. More preferably, the aromatic compound or the aromatic moiety of the both aromatic and aliphatic compound comprises one or two C₆ rings, the two rings being either separate or being present in condensed form. Aromatic compounds to be mentioned in particular are benzene, naphthalene and/or biphenyl and/or bipyridyl and/or pyridine. The aromatic compound or the aromatic moiety of the both aromatic and aliphatic compound can have one or alternatively more than one substituent that does not coordinate to the metal ion. Suitable substituents include —OH, a nitro group, an amino group or an alkyl or alkoxy group.

Examples to be mentioned within the scope of the present invention of dicarboxylic acids are 1,4-butanedicarboxylic acid, 4-oxopyran-2,6-dicarboxylic acid, 1,6-hexanedicarboxylic acid, decanedicarboxylic acid, 1,8-heptadecanedicarboxylic acid, 1,9-heptadecanedicarboxylic acid, heptadecanedicarboxylic acid, acetylenedicarboxylic acid, 1,2-benzenedicarboxylic acid, 2,3-pyridinedicarb oxylic acid, pyridine-2,3-dicarboxylic acid, 1,3-butadiene-1,4-dicarboxylic acid, 1,4-benzenedicarboxylic acid, p-benzenedicarboxylic acid, imidazole-2,4-dicarboxylic acid, 2-methylquinoline-3,4-dicarboxylic acid, quinoline-2,4-dicarboxylic acid, quinoxaline-2,3-dicarboxylic acid, 6-chloroquinoxaline-2,3-dicarboxylic acid, 4,4′-diaminophenylmethane-3,3′-dicarboxylic acid, quinoline-3,4-dicarboxylic acid, 7-chloro-4-hydroxyquinoline-2,8-dicarboxylic acid, diimidedicarboxylic acid, pyridine-2,6-dicarboxylic acid, 2-methylimidazole-4,5-dicarboxylic acid, thiophene-3,4-dicarboxylic acid, 2-isopropylimidazole-4,5-dicarboxylic acid, tetrahydropyran-4,4-dicarboxylic acid, perylene-3,9-dicarboxylic acid, perylenedicarboxylic acid, Pluriol E 200-dicarboxylic acid, 3,6-dioxaoctanedicarboxylic acid, 3,5-cyclohexadiene-1,2-dicarboxylic acid, octanedicarboxylic acid, pentane-3,3-carboxylic acid, 4,4′-diamino-1,1′-diphenyl-3,3′-dicarboxylic acid, 4,4′-diaminodiphenyl-3,3′-dicarboxylic acid, benzidine-3,3′-dicarboxylic acid, 1,4-bis-(phenylamino)benzene-2,5-dicarboxylic acid, 1,1′-dinaphthyl-8,8′-dicarboxylic acid, 7-chloro-8-methylquinoline-2,3-dicarboxylic acid, 1-anilinoanthraquinone-2,4′-dicarboxylic acid, polytetrahydrofuran-250-dicarboxylic acid, 1,4-bis(carboxymethyl)piperazine-2,3-dicarboxylic acid, 7-chloroquinoline-3,8-dicarboxylic acid, 1-(4-carboxyl)phenyl-3-(4-chloro)phenylpyrazoline-4,5-dicarboxylic acid, 1,4,5,6,7,7,-hexachloro-5-norbornene-2,3-dicarboxylic acid, phenylindanedicarboxylic acid, 1,3-dibenzyl-2-oxoimidazolidine-4,5-dicarboxylic acid, 1,4-cyclohexanedicarboxylic acid, naphthalene-1,8-dicarboxylic acid, 2-benzoylbenzene-1,3-dicarboxylic acid, 1,3-dibenzyl-2-oxoimidazolidine-4,5-cisdicarboxylic acid, 2,2′-biquinoline-4,4′-dicarboxylic acid, pyridine-3,4-dicarboxylic acid, 3,6,9-trioxaundecanedicarboxylic acid, o-hydroxybenzophenonedicarboxylic acid, Pluriol E 300-dicarboxylic acid, Pluriol E 400-dicarboxylic acid, Pluriol E 600-dicarboxylic acid, pyrazole-3,4-dicarboxylic acid, 2,3-pyrazinedicarboxylic acid, 5,6-dimethyl-2,3-pyrazinedicarboxylic acid, 4,4′-diaminodiphenyletherdiimidedicarboxylic acid, 4,4′-diaminodiphenylmethanediimidedicarboxylic acid, 4,4′-diaminodiphenylsulfonediimidedicarboxylic acid, 2,6-naphthalenedicarboxylic acid, 1,3-adamantanedicarboxylic acid, 1,8-naphthalenedicarboxylic acid, 2,3-naphthalenedicarboxylic acid, 8-methoxy-2,3-naphthalenedicarboxylic acid, 8-nitro-2,3-naphthalenedicarboxylic acid, 8-sulfo-2,3-naphthalenedicarboxylic acid, anthracene-2,3-dicarboxylic acid, 2′-3′-diphenyl-p-terphenyl-4,4″-dicarboxylic acid, diphenylether-4,4′-dicarboxylic acid, imidazole-4,5-dicarboxylic acid, 4(1H)-oxothiochromene-2,8-dicarboxylic acid, 5-t-butyl-1,3-benzenedicarboxylic acid, 7,8-quinolinedicarboxylic acid, 4,5-imidazoledicarboxylic acid, 4-cyclohexene-1,2-dicarboxylic acid, hexatriacontanedicarboxylic acid, tetradecanedicarboxylic acid, 1,7-heptanedicarboxylic acid, 5-hydroxy-1,3-benzenedicarboxylic acid, pyrazine-2,3-dicarboxylic acid, furan-2,5-dicarboxylic acid, 1-nonene-6,9-dicarboxylic acid, eicosenedicarboxylic acid, 4,4′-dihydroxydiphenylmethane-3,3′-dicarboxylic acid, 1-amino-4-methyl-9,10-dioxo-9,10-dihydroanthracene-2,3-dicarboxylic acid, 2,5-pyridinedicarboxylic acid, cyclohexene-2,3-dicarboxylic acid, 2,9-dichlorofluorubin-4,11-dicarboxylic acid, 7-chloro-3-methylquinoline-6,8-dicarboxylic acid, 2,4-dichlorobenzophenone-2′,5′-dicarboxylic acid, 1,3-benzenedicarboxylic acid, 2,6-pyridinedicarboxylic acid, 1-methylpyrrole-3,4-dicarboxylic acid, 1-benzyl-1H-pyrrole-3,4-dicarboxylic acid, anthraquinone-1,5-dicarboxylic acid, 3,5-pyrazoledicarboxylic acid, 2-nitrobenzene-1,4-dicarboxylic acid, heptane-1,7-dicarboxylic acid, cyclobutane-1,1-dicarboxylic acid, 1,14-tetradecanedicarboxylic acid, 5,6-dehydronorbornane-2,3-dicarboxylic acid or 5-ethyl-2,3-pyridinedicarboxylic acid, of tricarboxylic acids are

2-hydroxy-1,2,3-propanetricarboxylic acid, 7-chloro-2,3,8-quinolinetricarboxylic acid, 1,2,4-benzenetricarboxylic acid, 1,2,4-butanetricarboxylic acid, 2-phosphono-1,2,4-butanetricarboxylic acid, 1,3,5-benzenetricarboxylic acid, 1-hydroxy-1,2,3-propanetricarboxylic acid, 4,5-dihydro-4,5-dioxo-1H-pyrrolo[2,3-F]quinoline-2,7,9-tricarboxylic acid, 5-acetyl-3-amino-6-methylbenzene-1,2,4-tricarboxylic acid, 3-amino-5-benzoyl-6-methylbenzene-1,2,4-tricarboxylic acid, 1,2,3-propanetricarboxylic acid or aurinetricarboxylic acid, or of tetracarboxylic acids are

1,1-dioxide-perylo[1,12-BCD]thiophene-3,4,9,10-tetracarboxylic acid, perylenetetracarboxylic acids such as perylene-3,4,9,10-tetracarboxylic acid or perylene-1,12-sulfone-3,4,9,10-tetracarboxylic acid, butanetetracarboxylic acids such as 1,2,3,4-butanetetracarboxylic acid or meso-1,2,3,4-butanetetracarboxylic acid, decane-2,4,6,8-tetracarboxylic acid, 1,4,7,10,13,16-hexaoxacyclooctadecane-2,3,11,12-tetracarboxylic acid, 1,2,4,5-benzenetetracarboxylic acid, 1,2,11,12-dodecanetetracarboxylic acid, 1,2,5,6-hexanetetracarboxylic acid, 1,2,7,8-octanetetracarboxylic acid, 1,4,5,8-naphthalenetetracarboxylic acid, 1,2,9,10-decanetetracarboxylic acid, benzophenonetetracarboxylic acid, 3,3′,4,4′-benzophenonetetracarboxylic acid, tetrahydrofurantetracarboxylic acid or cyclopentanetetracarboxylic acids such as cyclopentane-1,2,3,4-tetracarboxylic acid.

Most especially preferred within the scope of the present invention is the use, where suitable, of mono-, di-, tri-, tetra- or polynuclear aromatic di, tri- or tetracarboxylic acids. The metal salt may be any metal salt suitable for forming a metal-organic framework. In particular, in combination with the abovementioned ligands. Typically, the metal salt is selected from the group consisting of Groups 1 through 16 of the Periodic Table, as those groups are defined by IUPAC, including the lanthanides and the actinides and blends thereof. Metal ions can have any valence the metal can exhibit. Preferably, a metal is selected from the group consisting of transition metals, the alkaline earth metals, lanthanides and blends thereof. Preferably, the metal is selected from the group consisting of nickel, magnesium, bismuth, gallium, cobalt, copper, zinc, aluminium, scandium, yttrium, iron, cadmium and blends thereof. More preferably metal ions are selected from the group consisting of Ni²⁺, Mg²⁺, Bi³⁺, Ga³⁺, Co²⁺, Cu²⁺, Zn²⁺, Al³⁺, Sc³⁺, Y³⁺, Fe³⁺, Cd²⁺. The skilled person will be able to select appropriate counter ions depending on the target metal-organic framework and ligand. Preferred counter ions include, but are not limited to, nitrates, acetates, halides, and oxides.

Metal-organic frameworks which may be synthesised using the continuous flow process of the invention include, but are not limited to, MIL-53 (Al), Al-MIL-53-NH₂, MIL-96, MIL-100 (Al), MIL-110, MIL-69, CAU-1, MIL-122, DUT-5, MIL118, MIL-120, MIL-121, MIL-122, MIL-101 (Al), MIL-53 (Sc), Sc₂BDC₃ also known as Sc₂(O₂CC₆H₄CO₂)₃, Sc₂(NH₂-BDC)₃, Sc₂(NO₂-BDC)₃, RPF-12, RPF-13, MIL-88 (Sc), MIL-100 (Sc), NOTT-400, NOTT-401, {[Sc₃O(1,4-benzene-dicarboxylate)₃(H₂O)₃].Cl_{0.5}(OH)_{0.5}(DMF)₄(H₂O)₃}, {[Sc₃O(5′-(4-carboxylatophenyl)-[1,1′:3′,1″-terphenyl]-4,4″-dicarboxylate))₂(H₂O)₃](OH)(H₂O)₅(DMF)}, Sc₂(O₂CC₂H₄CO₂)_{2.5}(OH), Sc₂(C₄O₄)₃, RPF-14, MIL-78, MIL-53 (Fe), MIL-100 (Fe), MOF-235, Fe-BTC, MIL-45 (Fe), MOF-74 (Fe), COP-1 (Zn), UMCM-1, UMCM-2, MOF-5, MOF-74(Zn), MOF-177, IRMOF-1, IRMOF-2, IRMOF-3, IRMOF-4, IRMOF-5, IRMOF-6, IRMOF-7, IRMOF-8, IRMOF-9, IRMOF-10, IRMOF-11, IRMOF-12, IRMOF-13, IRMOF-14, IRMOF-15, IRMOF-16, UTSA-36, MCF-27, MOF-38, CAU-5, MOF-205, DUT-6, CPO-27 (Zn), TIF-2 (Zn), TIF-A1 (Zn), TIF-A2 (Zn), ZBIF-1 (Zn), ZIF-1, ZIF-10, ZIF-2, ZIF-3, ZIF-4, ZIF-60, ZIF-61, ZIF-62, ZIF-64, ZIF-70, ZIF-76, ZIF-67, NOTT-100, NOTT-101, NOTT-102, NOTT-103, NOTT-104, NOTT-105, NOTT-106, NOTT-107, NOTT-108, NOTT-109, NOTT-110, NOTT-111, NOTT-112, NOTT-113, NOTT-114, NOTT-115, NOTT-116, NOTT-119, NOTT-140, HKUST-1 also known as Cu₃(BTC)₂(H₂O)₃, NENU-27, MOF-505, MOF-14, MOF-143, DUT-6 (Zn), MOF-39, UMCM-1, UMCM-2. NOTT-200, NOTT-201, NOTT-204, NOTT-205, NOTT-206, NOTT-207, NOTT-208, NOTT-209, NOTT-210, NOTT-211, NOTT-212, NOTT-213, MIL-122, CPM-20, CPM-18 (In), SCIF-1, MIL-53 (Cr), MIL-101 (Cr), Cr₃(BTC)₂, CPM-20 (Co), UIO-66 (Zr), CPM-24 (Co), MIL-45 (Co), CPO-27 (Co), MOF-74 (Co), MOF-501 (Co), MIL-53 (Ga), CP0-1 (Cd), Mn(BDC)(H₂O)₂, Ni₃(BTC)₂.12H₂O, DUT-8 (Ni), Mg₃(NDC)₃, CPO-27 (Mg), CPO-27 (Ni), MOF-74 (Mg), MOF-74 (Ni), CPF-1, SNU-M11 (Ni), NOTT 300 (Al), NOTT 300 (Ga), [Zn₂(4′,5′-bis(4-carboxylatophenyl)-[1,1′:2′,1″-terphenyl]-4,4″-dicarboxylate)]. It will be appreciated that the present invention may, in theory, be used to synthesise any metal-organic framework.

Typically, the solvent, preferably water and/or ethanol, is preheated to a temperature of at least about 200° C., preferably above 250° C., more preferably at least about 300° C. Preferably, the solvent is supercritical or near critical water, more preferably near critical water. The critical point of water is 374° C. and 22 MPa. For the purposes of the invention, near critical water is understood to be water at a temperature of from about 200° C. to about 374° C. and at a pressure of from about 1.55 MPa to about 22 MPa.

Preferably, the water and/or ethanol is at a temperature such that the ligand is soluble therein. Preferably, the water and/or water-ethanol mixture is at a temperature of from about 200° C. to about 500° C., preferably from about 220° C. to about 374° C., from about 240° C. to about 280° C. is particularly preferred. Where ethanol alone is used preferably, the ethanol is at a temperature of from about 100° C. to about 400° C., preferably from about 125° C. to about 300° C., from about 200° C. to about 250° C. is particularly preferred. Typically, the water and/or ethanol are at a pressure of from about 1.55 MPa to about 23 MPa.

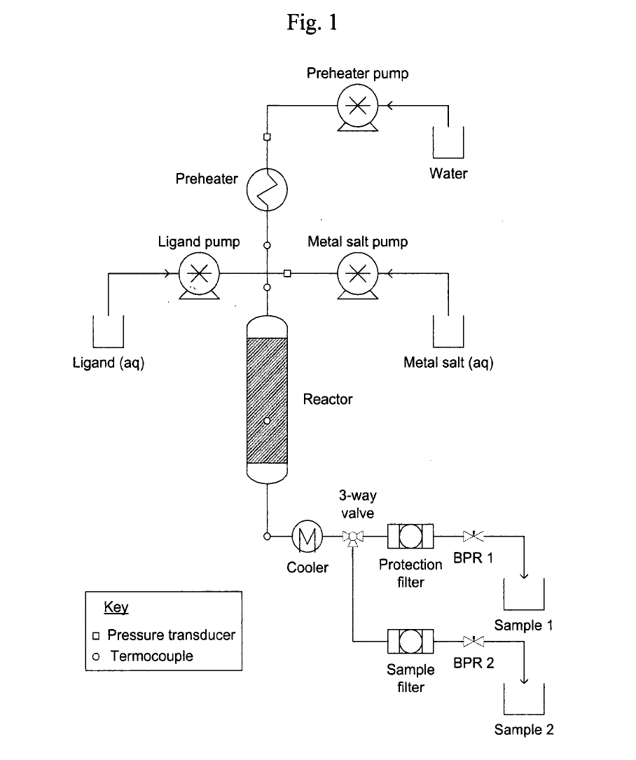

FIG. 1 shows a schematic of a process according to the invention. Aqueous and/or ethanolic solutions of a salt and a ligand are pumped by means of HPLC pumps (e.g. Gilson 306, 10 ml pump heads). The ligand and salt are mixed with another stream of water preheated to at least about 300° C. before entering the reactor (piping available from Swagelok). The temperature of the reactor is kept constant using resistance heaters and a temperature controller (Eurotherm 2216L). Once the reaction is complete, the mixture is cooled downstream by a heat exchanger. The solid product is recovered by a Tee filter. After filtration the liquid by-product stream enters a back pressure regulator (BPR) (Tescom, model no. 26-1762-24-043). Filtration is used to avoid malfunctioning of the BPR. Two identical sets of filters and BPR are installed. Using a three-way valve the flow can be directed to either of them. One of the filters is used to collect the sample for a determined time and the other is to protect the BPR retaining any solid while waiting the steady state is reached. This experimental array allows collecting samples when steady state is reached.

The present invention also provides a method for the treatment of a metal-organic framework to increase the available internal surface area and/or porosity typically comprising the steps of:
a. providing a metal-organic framework formed by reacting a ligand and a metal salt;
b. introducing a supercritical fluid into the metal-organic framework; and
c. removing the supercritical fluid;
   characterised in that unreacted ligand is soluble in the supercritical fluid.

A supercritical fluid is understood to be any substance at a temperature and pressure above its critical point. Above the critical point of a substance distinct liquid and gas phases do not exist.

Typically, the supercritical fluid is an organic supercritical fluid. Typically, it is chosen from the list consisting of ethanol, methanol, propanol, butanol, acetone, chloroform, fluoroform, CF₃CH₂F, ammonia, dimethyl ether, diethyl ether. Supercritical ethanol (scEtOH) is particularly preferred. The critical point of ethanol is 241° C. and 6.3 MPa. Preferably, the supercritical fluid is used in combination with a cosolvent. Typically, the cosolvent is a supercritical fluid. Any one of the above-listed supercritical fluids may be used as a cosolvent. A particularly preferred cosolvent is supercritical carbon dioxide. Supercritical carbon dioxide may only be used as a cosolvent because ligands, and reaction by-products such as their carboxylic acid analogues, tend not to be soluble therein. Supercritical carbon dioxide is however useful for removing any solvent left over from the synthesis of the metal-organic framework. The use of supercritical ethanol with supercritical carbon dioxide cosolvent is particularly preferred. Such a combination is useful in the removal of solvent and ligand/carboxylic acid analogues of the ligand, particularly when the metal-organic framework has been synthesised using DMF.

It will be appreciated that the method of the invention may be used with any metal-organic framework. Metal-organic frameworks that may be treated with the above-cited process include, but are not limited to, MIL-53 (Al), Al-MIL-53-NH₂, MIL-96, MIL-100 (Al), MIL-110, MIL-69, CAU-1, MIL-122, DUT-5, MIL118, MIL-120, MIL-121, MIL-122, MIL-101 (Al), MIL-53 (Sc), Sc₂BDC₃ also known as Sc₂(O₂CC₆H₄CO₂)₃, Sc₂(NH₂—BDC)₃, Sc₂(NO₂—BDC)₃, RPF-12, RPF-13, MIL-88 (Sc), MIL-100 (Sc), NOTT-400, NOTT-401, {[Sc₃O(1,4-benzene-dicarboxylate)₃(H₂O)₃].Cl_{0.5}(OH)_{0.5}(DMF)₄(H₂O)₃}, {[Sc₃O(5′-(4-carboxylatophenyl)-[1,1′:3′,1″-terphenyl]-4,4″-dicarboxylate))₂(H₂O)₃](OH)(H₂O)₅(DMF)}, Sc₂(O₂CC₂H₄CO₂)₂₅(OH), Sc₂(C₄O₄)₃, RPF-14, MIL-78, MIL-53 (Fe), MIL-100 (Fe), MOF-235, Fe-BTC, MIL-45 (Fe), MOF-74 (Fe), COP-1 (Zn), UMCM-1, UMCM-2, MOF-5, MOF-74(Zn), MOF-177, IRMOF-1, IRMOF-2, IRMOF-3, IRMOF-4, IRMOF-5, IRMOF-6, IRMOF-7, IRMOF-8, IRMOF-9, IRMOF-10, IRMOF-11, IRMOF-12, IRMOF-13, IRMOF-14, IRMOF-15, IRMOF-16, UTSA-36, MCF-27, MOF-38, CAU-5, MOF-205, DUT-6, CPO-27 (Zn), TIF-2 (Zn), TIF-A1 (Zn), TIF-A2 (Zn), ZBIF-1 (Zn), ZIF-1, ZIF-10, ZIF-2, ZIF-3, ZIF-4, ZIF-60, ZIF-61, ZIF-62, ZIF-64, ZIF-70, ZIF-76, ZIF-67, NOTT-100, NOTT-101, NOTT-102, NOTT-103, NOTT-104, NOTT-105, NOTT-106, NOTT-107, NOTT-108, NOTT-109, NOTT-110, NOTT-111, NOTT-112, NOTT-113, NOTT-114, NOTT-115, NOTT-116, NOTT-119, NOTT-140, HKUST-1 also known as Cu₃(BTC)₂(H₂O)₃, NENU-27, MOF-505, MOF-14, MOF-143, DUT-6 (Zn), MOF-39, UMCM-1, UMCM-2. NOTT-200, NOTT-201, NOTT-204, NOTT-205, NOTT-206, NOTT-207, NOTT-208, NOTT-209, NOTT-210, NOTT-211, NOTT-212, NOTT-213, MIL-122, CPM-20, CPM-18 (In), SCIF-1, MIL-53 (Cr), MIL-101 (Cr), Cr₃(BTC)₂, CPM-20 (Co), UIO-66 (Zr), CPM-24 (Co), MIL-45 (Co), CPO-27 (Co), MOF-74 (Co), MOF-501 (Co), MIL-53 (Ga), CP0-1 (Cd), Mn(BDC)(H₂O)₂, Ni₃(BTC)₂.12H₂O, DUT-8 (Ni), Mg₃(NDC)₃, CPO-27 (Mg), CPO-27 (Ni), MOF-74 (Mg), MOF-74 (Ni), CPF-1, SNU-M11 (Ni), NOTT-300 (Al), NOTT-300 (Ga), [Zn₂(4′,5′-bis(4-carboxylatophenyl)-[1,1′:2′,1″-terphenyl]-4,4″-dicarboxylate)].

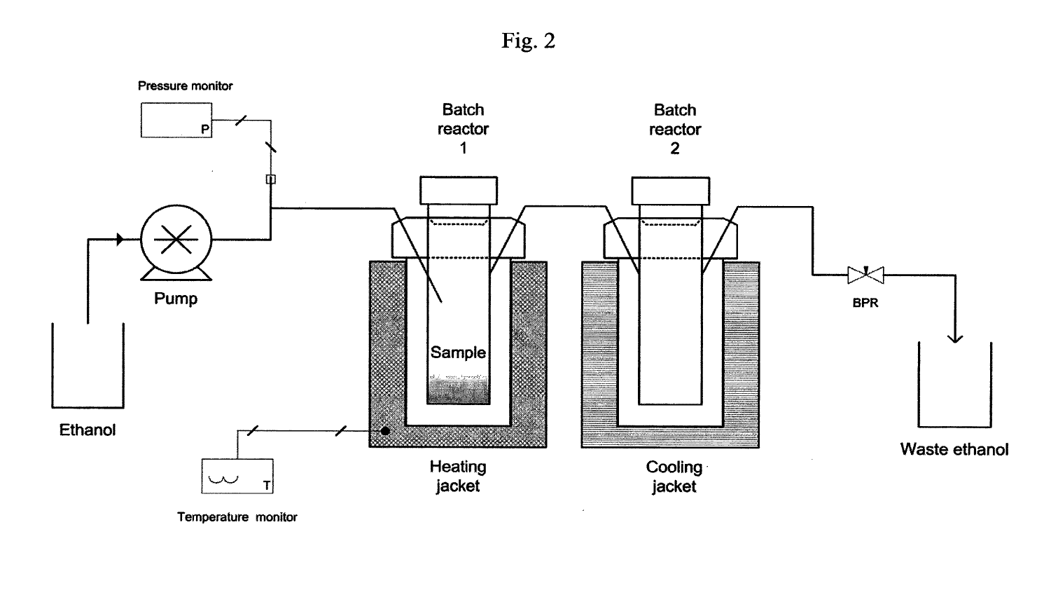

FIG. 2 provides a schematic of the setup for using supercritical ethanol (T_{c}=241° C., P_{c}=6.3 MPa) to extract ligand from a metal-organic framework. A sample of metal-organic framework (e.g. MIL-53(Al)) is loaded into the first reactor and both reactors are filled with ethanol before sealing. Supercritical ethanol is pumped (0.5 ml min⁻¹) into the first reactor and flows over the sample. The flow then goes into a second reactor which is cooled before passing through a back pressure regulator (BPR) which maintains pressure of 100 bar. The reactor heating block was heated to approximately 260° C. (ramp rate 520° C. min⁻¹). The internal reactor temperature is approximately 252° C. When cooling down pressure and flow is maintained until cool, the system is then slowly depressurised.

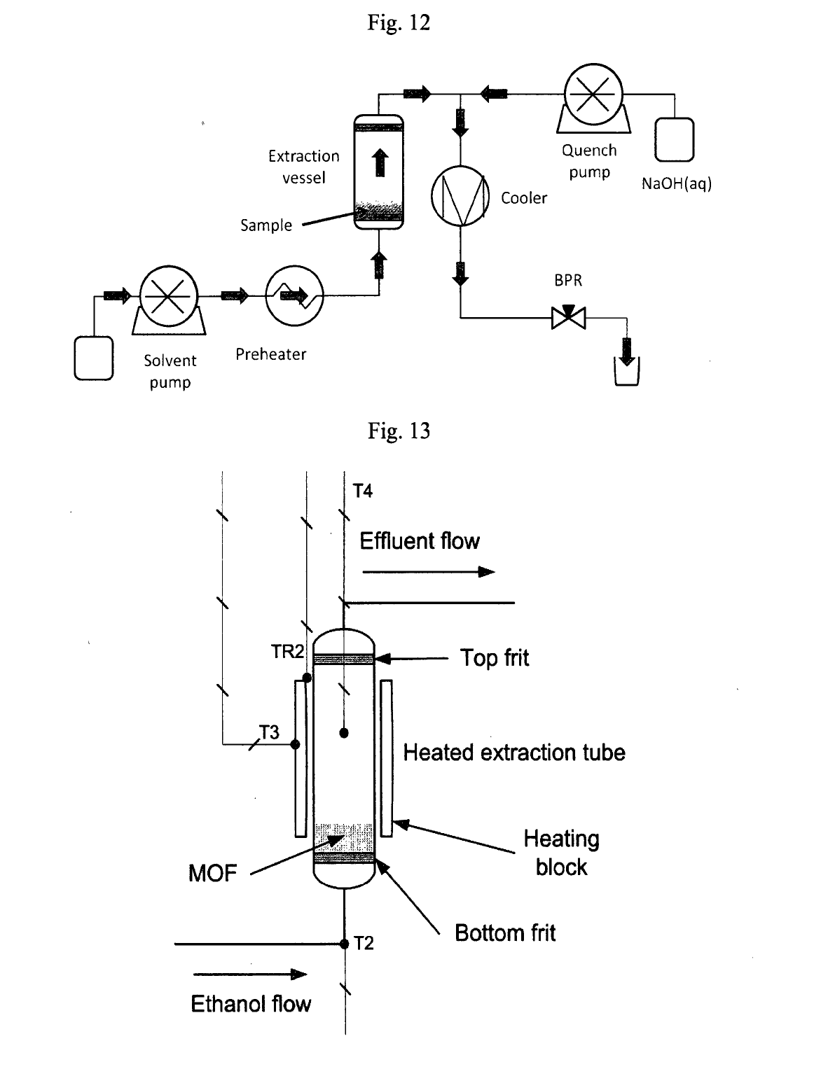

FIG. 12 provides a schematic of an alternative arrangement to that shown in FIG. 2 of the method for treating a metal-organic framework according to the invention. FIG. 13 provides a close-up of the extraction vessel. The extraction vessel typically comprises an extraction tube, a heating block with band heater, and reducing union at each end. For each extraction, the extraction vessel must be removed, the sample loaded into the extraction tube, and the vessel refitted to the rig. The sample (MOF) is held in the extraction tube by frits (2 μm sintered steel) located in the reducing unions at each end of the extraction vessel.

The solvent is pumped by the solvent pump, typically an HPLC pump (e.g. Gilson 302, 10 ml pump head) into a preheater. The temperature of the preheater is typically maintained by a temperature controller (Eurotherm 3216L) with a built-in trip (West 6700+). The preheater is constructed by coiling ⅛″ tube around a brass heating block. The temperature controller powers a heating band around the tube coil on the outside and a heating cartridge within the heating block. The temperature controller is typically connected to a K-type thermocouple within a predrilled hole in the heating block. The trip is connected to a second K-type thermocouple placed in contact with the band heater and the heating block.

As the flow leaves the preheater the temperature is measured by an internal K-type thermocouple. The flow then enters the extraction tube which is kept at a constant temperature by a temperature controller (Eurotherm 2216L) with a built-in trip (West 6700) which powers a resistive heating band around an aluminium heating block. The temperature controller is connected to a K-type thermocouple within a predrilled hole in the aluminium heating block. The trip is connected to a second K-type thermocouple in contact with the band heater and heating block, and in the event of overheating, this trip also stops power to the preheater controller. The temperature is measured inside the extraction tube by an internal K-type thermocouple before the flow leaves the extraction tube and is mixed with a quench flow before it enters the heat exchanger. A quench flow is pumped by the quench pump, typically an HPLC pump (e.g. Gilson 302, 5.0 ml pump head). The cooled flow then passes out through the backpressure regulator (BPR). The NaOH quench flow ensures a homogeneous mixture of sodium terephthalate is present in the effluent flow. This is advantageous for online or offline analysis of the ligand concentration and prevents precipitation of the unreacted terephthalic acid in the BPR. Precipitation in this way would impede the BPR. The heat exchange unit cools the effluent for release and prevents damage occurring to the BPR unit as a result of overheating. An alternative to the use of the quench pump to prevent malfunction of the BPR would be to install a filter before the before BPR.

The present invention also provides a method for synthesising a metal-organic framework using recycled unreacted ligand comprising the steps of:
a. providing a first metal-organic framework formed by reacting a ligand and a metal salt;
b. extracting any unreacted ligand present in the first metal-organic framework; and
c. using said extracted unreacted ligand to synthesise a second metal-organic framework.

Typically, the first and/or second metal-organic framework is synthesised using the process of the invention, although other processes for synthesising the metal-organic frameworks could also be employed, such as using microwave reactors or solvothermal batch processes.

Typically, the unreacted ligand is extracted using a supercritical fluid, preferably ethanol. Metal-organic frameworks synthesised or treated using the methods and processes of the invention are particularly useful in gas storage (e.g. methane, hydrogen and carbon dioxide), purification of gases, gas separation, catalysis and in sensors. This is in part due to their improved porosity and/or increased availability of the internal surface area and/or gas storage properties.

The continuous flow process offers reaction times much shorter than is possible with batch reactors, making the process more suitable for large scale manufacturing, while when using water and/or ethanol they also having better green credentials. This is a significant improvement over batch solvothermal systems using solvents such as DMF.

Currently metal-organic frameworks, such as MIL-53(Al), are sold at [symbol] 2660-3850 for 500 g. This pricing level is too high for their utilisation in many applications. A more productive process should reduce this cost. The space-time-yield for the continuous flow process of the invention is typically at least about 500 kg m⁻³ d⁻¹, preferably at least about 1200 kg m⁻³ d⁻¹. Reported space-time-yield values (kg m⁻³ d⁻¹) for commercially available metal-organic frameworks are between 20 and 300 with MIL-53 (Al) at 160.

### Examples

### Synthesis

Samples were prepared using two distinct processes, the first being batch and the second a continuous flow process.

### Batch

In batch the reaction of Al(NO₃)₃ with terephthalic acid (H₂L) in a reactor at 250° C. for 10 minutes yielded the metal-organic framework material MIL-53(Al). A ratio of 2:3 for Al(NO₃)₃ to terephthalic acid was used with concentrations of 0.04 mol dm⁻³ and 0.06 mol dm⁻³, respectively. The synthesis of MIL-53 (Al) has been confirmed by powder X-ray diffraction and further product characterisation using gas adsorption and TGA.

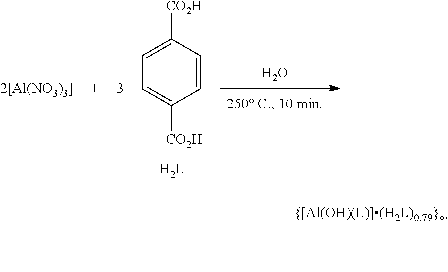 [SMILES: O=C(O)c1ccc(C(=O)O)cc1 ; O]

### Reaction Scheme for Batch Reaction

Samples were produced at 150° C., 200° C., 250° C., and 300° C.

### Continuous Flow

### Water Solvent

Metal-organic frameworks according to the invention were synthesised using a tubular 316 stainless steel continuous flow reactor (ID 0.370 inches, reactor volume 20.8 ml). A schematic of the experimental device is shown in FIG. 1.

Aqueous solutions of aluminium nitrate (0.05 mol dm⁻³) and disodium terephthalate (0.05 mol dm⁻³) were both pumped at a flow rate of 1 ml min⁻¹ by means of HPLC pumps (Gilson 306, 10 ml pumpheads). They were mixed with another stream of water pumped at a flow rate of 1 ml min⁻¹ then preheated to 300° C. before entering the reactor (piping available from Swagelok, outside diameter 0.50 inches, wall thickness 0.065 inches). The total pumped flow rate entering the reactor was approximately 3 ml min⁻¹. The temperature of the reactor is kept constant using resistance heaters and a temperature controller (Eurotherm 2216L). Once the reaction is complete, the mixture is cooled downstream by a heat exchanger. The solid product was recovered by a Tee filter (0.5 μm). After filtration the liquid by-product stream enters the back pressure regulator (BPR) (Tescom, model no. 26-1762-24-043). Filtration was used to avoid malfunctioning of the BPR. Two identical sets of filters and BPRs were installed. Using a three-way valve the flow can be directed to either of them. One of the filters is called a sampling filter, used to collect the sample for a determined time and the other is a protection filter to protect the BPR retaining any solid whilst the steady state is reached. This experimental array allows collecting samples when steady state is reached.

The experimental procedure was as follows: Pumps were set to the desired flow rates and water is pumped through them. Pressure of the system, temperature of the preheater and the reactor were set to the desired values. When the temperature was stable, the streams were changed to metal salt and ligand solutions and the flow was passed through the Protection Filter for 20 min. Then the three way valve is switched to sampling filter and sample is collected for 20 min. After the collection time, the three way valve it is switched back to protection filter and new reaction conditions are set. The sampling filter was replaced by a clean one and after waiting until steady state was achieved at the new conditions; the procedure is repeated to collect a second sample. Samples were produced at 200° C., 225° C., 250° C., 275° C. and 300° C.

The samples synthesised according to the continuous flow process have free terephthalic acid, a by-product of the reaction, in the pores and powder. This was removed by washing with supercritical ethanol. FIG. 2 shows a schematic of the equipment used to wash the metal-organic framework to remove the unreacted ligand.

The reaction of aluminium nitrate and disodium terephthalate in continuous flow for 20 minutes at 250° C. yielded 0.4979 g (86%) of white powder.

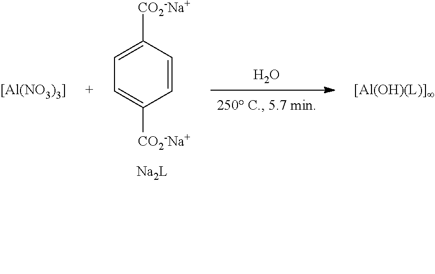

### Reaction Scheme for Continuous Flow Reaction

It was found that shorter reaction times were possible in continuous flow than in batch reactions. It has been shown that using a reaction time of approximately 5.7 minutes is sufficient to synthesise MIL-53 (Al).

The metal-organic framework MIL-53(Al) is known to exhibit a breathing effect and phase transitions induced by heating or guest species. Typically the as-synthesised phase (MIL-53 (Al) ta) contains free terephthalic acid trapped within the pores; this requires removal before gas adsorption experiments. The removal of terephthalic acid causes a phase change to a more open structure (MIL-53 (Al) op), this phase changes to the hydrated phase (MIL-53 (Al) hy), upon adsorption of water and the pores contract. The phase transition from the open to hydrated form is reversible by removal of the water through heating.

The following techniques were used for characterisation; powder X-ray diffraction (PXRD), in situ powder X-ray diffraction, volumetric gas adsorption, thermogravemetric analysis and elemental analysis.

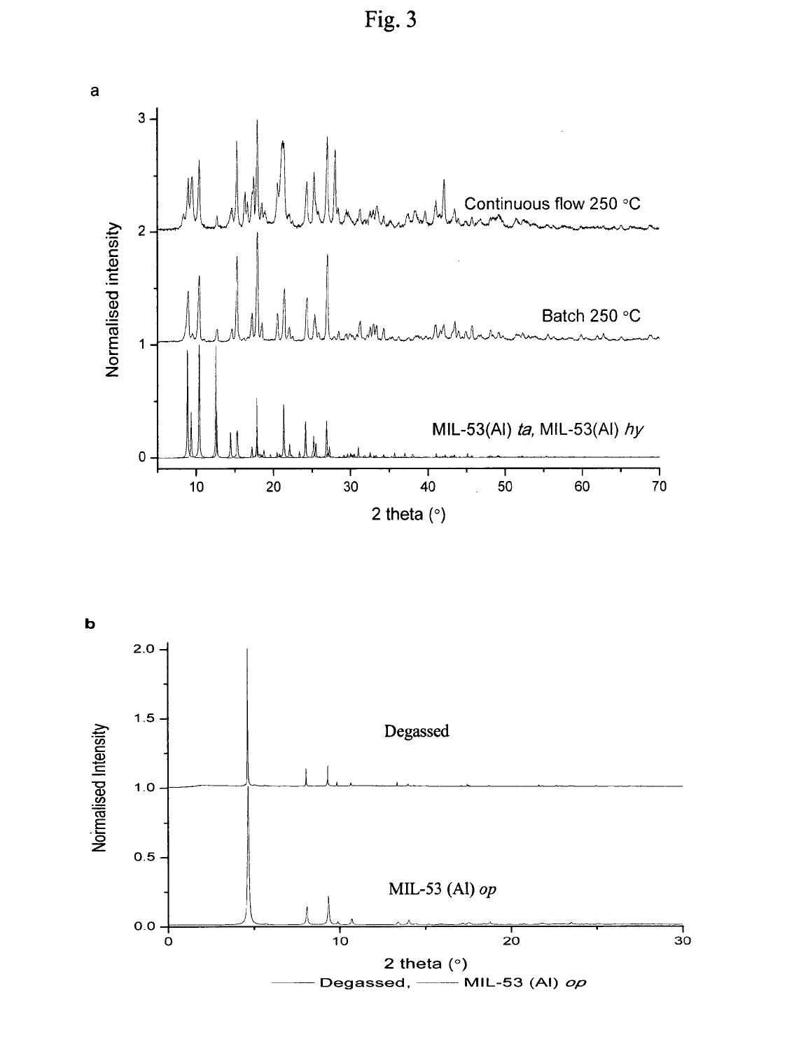

PXRD shows both the as-sythesised batch and continuous flow product are microcrystaline and match with two known MIL-53 (Al) phases (MIL-53 (Al) ta and MIL-53 (Al) op) (FIG. 3a). The PXRD for the batch reaction shows no significant peaks for impurities or unreacted starting material such as AlO(OH), by-products such as terephthalic acid or other metal-organic framework phases.

PXRD was carried out in-situ on a gas rig of I11 at Diamond Light Source (STFC Harwell Science and Innovation Campus) using a batch reaction sample (FIG. 3b). The pattern matches with the simulated pattern of MIL-53 (Al) op. The terephthalic acid occupying the pores was first removed by supercritical ethanol and the sample degassed (1 hour at 200° C. under vacuum).

FIG. 3 (a) shows a powder diffraction of as synthesised 250° C. batch sample and simulated powder diffraction patterns for MIL-53 (Al) ta and MIL-53 (Al) op. (b) In-stiu PXRD using a wavelength of 0.827107 Å with the degassed sample and simulated pattern of MIL-53(Al) op.

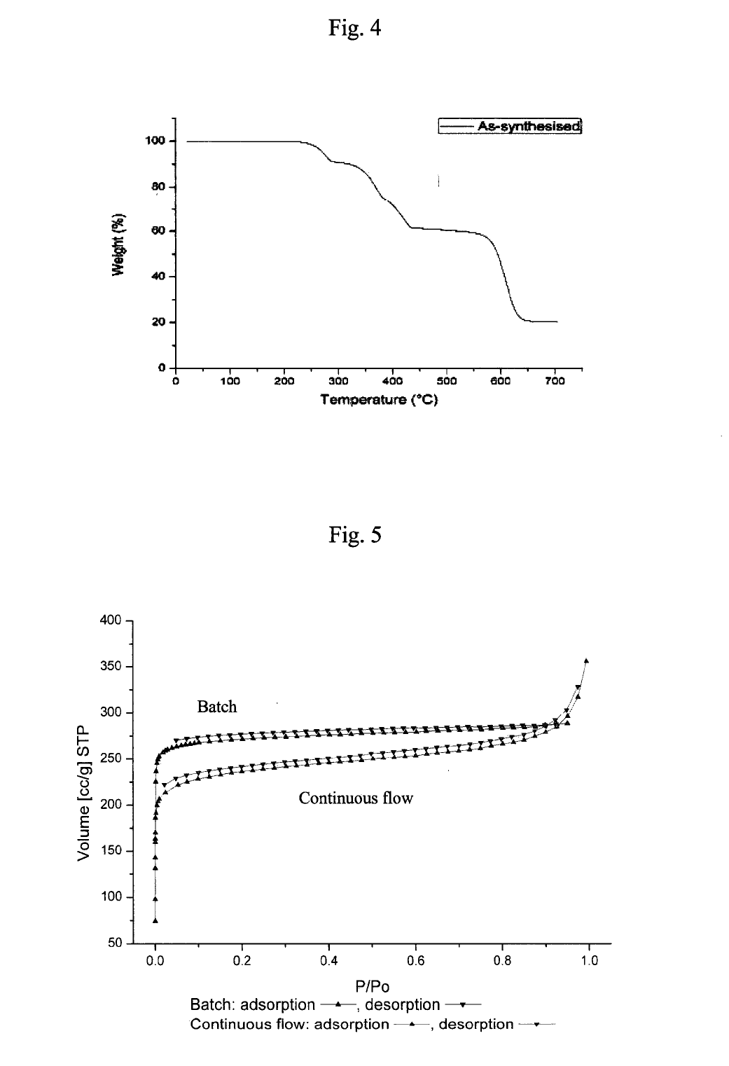

Thermogravimetric analysis (TGA) demonstrated the as-synthesised product has thermal stability up to 540° C. and contains 0.79 free terephthalic acid equivalents (FIG. 4). TGA was performed using Perkin Elmer Pryis 1 TGA (model no. R1R151 TGA, software Ver. 11.0.0.0449). A heating rate of 5° C. min⁻¹ was used from room temperature up to 700° C.

N₂ gas adsorption data recorded after removal of free terephthalic acid and activation on samples made at 250° C. in both batch and continuous flow. The isotherms are type I as expected for a microporous material. FIG. 5 shows a comparison of N₂ isotherms at 77K for batch and continuous flow product. The maximum uptake (at 0.95 P/P₀) for the batch and continuous flow sample was 289 cm⁻³ g⁻¹ and 296 cm⁻³ g⁻¹, respectively. The BET surface area of the batch and continuous flow sample was 1097 m² g⁻¹ and 913 m² g⁻¹, respectively. These measurements were performed using Quantachrome Autosorb-1 (model no. As1-GYTKXL11, software ver. 1.61).

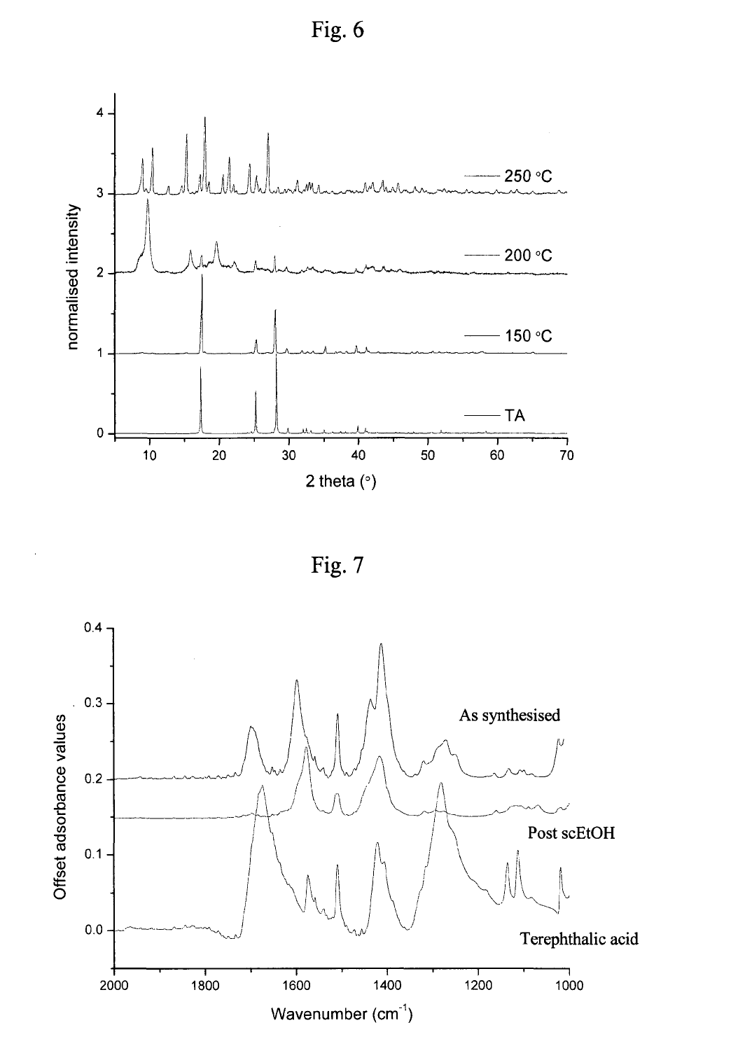

The effect of temperature on the product has been studied and has been shown to have large impact on batch reactions with short residence time. FIG. 6 shows PXRD of reaction product at different temperatures (150° C., 200° C. and 250° C.) and simulated pattern for terephthalic acid. At 250° C. the reaction is complete, however at 150° C. almost no MOF product is detected and much of the terephthalic acid is unreacted and recrystallised, at 200° C. the product is less crystalline and contains some unreacted terephthalic. These measurements were performed using Pananalytical X'Pert Pro diffractometer operated at 160 W (40 kV, 40 mA) for Cu Kα1 (λ=1.5406 Å).

### Synthesis of HKUST-1 in Continuous Flow

### Ethanol Solvent

HKUST-1 was synthesised according to the below reaction using the continuous flow method of the invention using ethanol as the solvent.

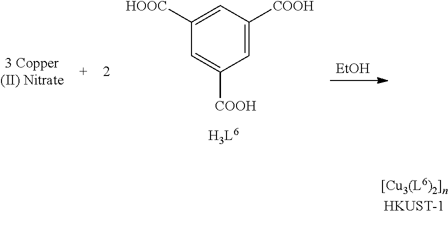 [SMILES: "EtOH" ; O=C(O)c1cc(C(=O)O)cc(C(=O)O)c1]

As both the copper (II) nitrate and the ligand are soluble in ethanol it is not necessary to use ligand salts. The continuous flow reaction was carried out using the equipment illustrated in FIG. 1 at 150° C. and 200° C., using a total flow rate of 3.0 ml min⁻¹ (preheated flow 1.0 ml min⁻¹, metal salt flow 1.0 ml min⁻¹ and ligand flow 1.0 ml min⁻¹), a feed concentration of 0.15 mol dm⁻³ and 0.10 mol dm⁻³ was used for the copper(II) nitrate and ligand respectively, the concentration in the reactor was 0.05 mol dm⁻³ and 0.03 mol dm⁻³ respectively. The pressure was kept constant at 7.5 MPa and the residence time for the reaction at 150° C. was 5.8 min (350 s) and at 200° C. 5.1 min (306 s). The samples were collected by filtration using a 0.5 μm filter.

After 15 minutes of collection the synthesis at 150° C. yielded a product weight of 0.103 g and after 15 minutes at 200° C. a product weight of 0.536 g was collected.

The product was characterised by PXRD, TGA, IR spectroscopies and N₂ adsorption isotherm.

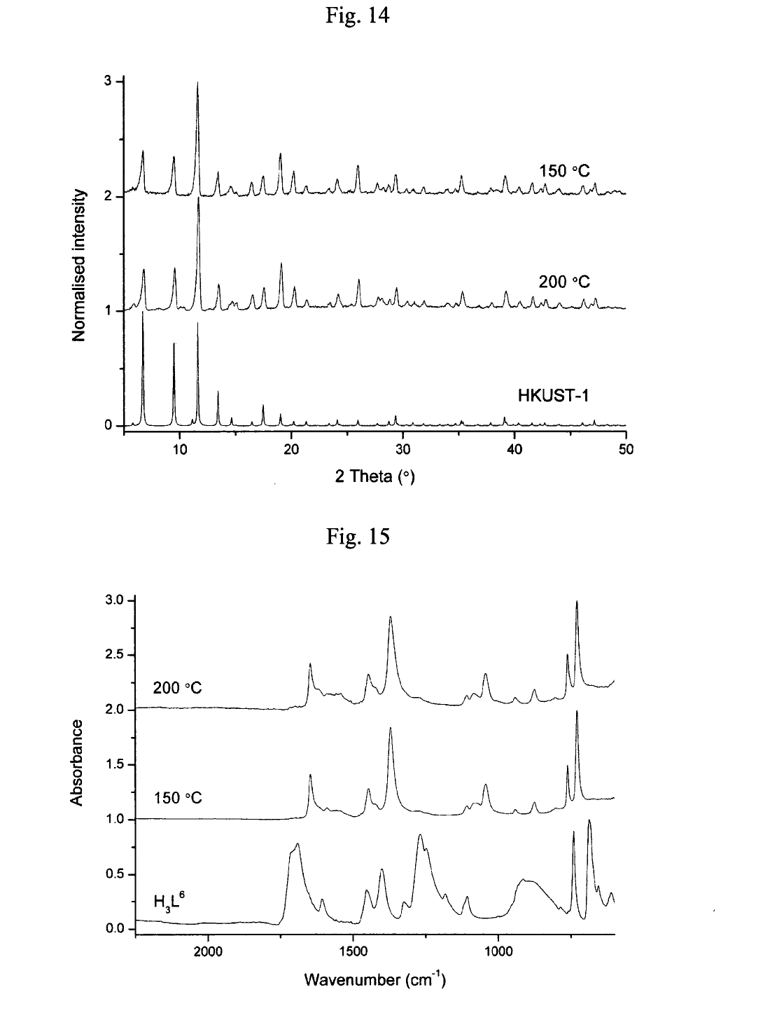

FIG. 14 shows a comparison of the PXRD of the as-synthesised samples from continuous flow synthesis at 150° C. and 200° C., to the simulated HKUST-1 pattern. The product of the continuous flow reaction is microcrystalline, phase pure, and matches the target phase HKUST-1.

FIG. 15 shows an IR of the as-synthesised HKUST-1 material produced by continuous flow synthesis at 150° C. and 200° C. Comparison of IR of the continuous flow products with the IR of the ligands shows that no uncoordinated ligand is present in the as-synthesised material.

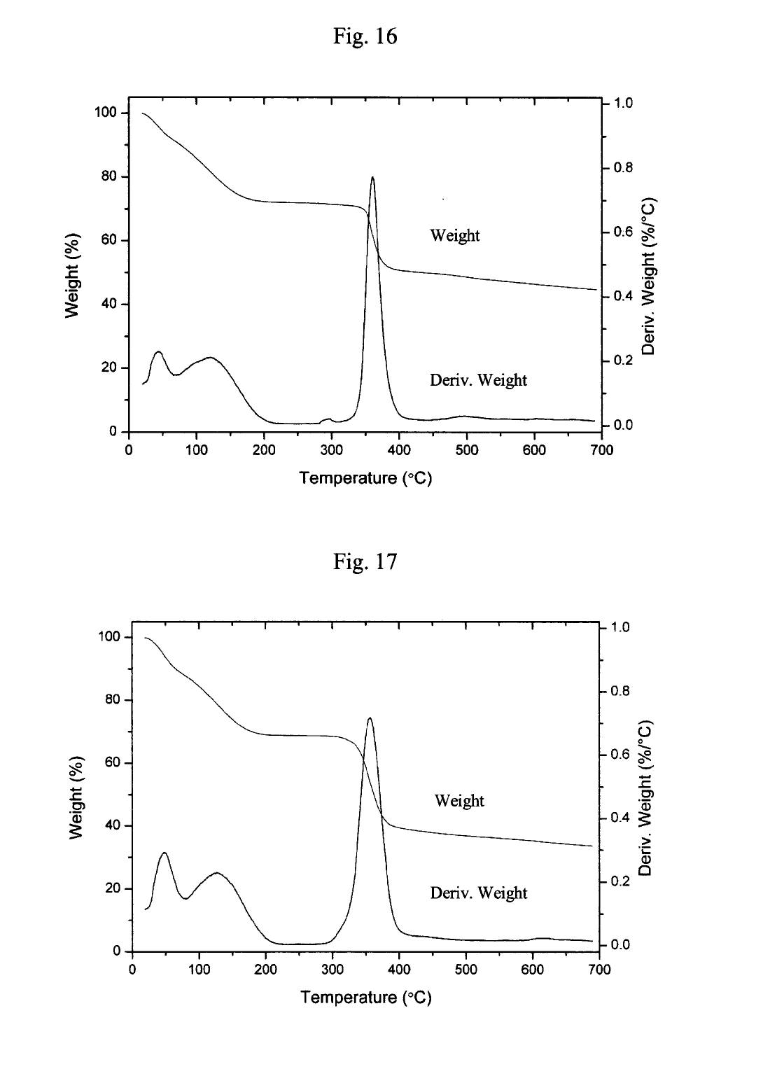

FIG. 16 shows a thermogravimetric analysis of as-synthesised HKUST-1 material from continuous flow reaction at 150° C. The continuous flow sample produced at 150° C. has three steps in the TGA: the first are from solvent loss and the third decomposition of the material. The first step of 8.9% between 20 to 70° C., is due to loss of solvent from within the pores; the second step of 18.9% between 70° C. and 220° C. is attributed to the loss of coordinated solvents. The third step of 21.5% between 320° C. and 450° C. corresponds to decomposition of the material.

FIG. 17 shows a thermogravimetric analysis (TGA) of as-synthesised HKUST-1 material from continuous flow reaction at 200° C. As with the other samples, the continuous flow sample produced at 200° C. exhibits the expected thermal behaviour of HKUST-1, with three steps. The first step of 11.4% between 20 to 70° C. being the result of solvent loss from within the pores, and the second step of 19.7% between 70° C. and 220° C. being attributed to the loss of coordinated solvents: giving a total solvent loss of 31.1%. The third step of 30.7% between 295° C. and 450° C. corresponds to decomposition of the material.

The TGA enables an estimate of the amounts of solvent contained within the material; this samples contained 31.25% weight, and therefore the yield of based on [Cu₃(C₆H₃(CO₂)₃)₂]n is 63%.

N₂ adsorption was used to assess the porosity of the material.

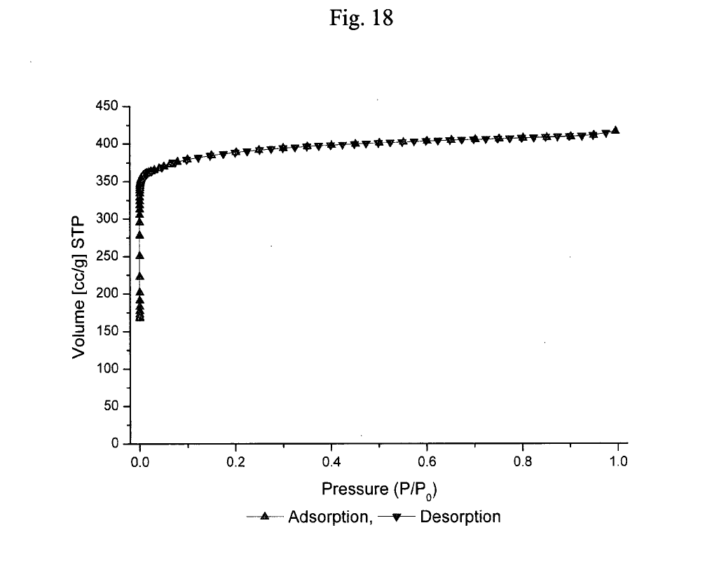

FIG. 18 shows an N₂ isotherm of 200° C. CF product HKUST-1 material from the reaction at 200° C. in continuous flow. The N₂ isotherm is type I, characteristic of a microporous material, and shows a maximum N₂ uptake of 416.9 cm³ g⁻¹ (0.95 P/P₀), a pore volume of 0.62 cm³ g⁻¹ and BET surface area of 1554 m² g⁻¹.

The space time yield is approximately 728 kg m⁻³ d⁻¹.

### Ligand Extraction Method

FIG. 2 provides a schematic of the setup for supercritical ethanol (T_{c}=241° C., P_{c}=6.3 MPa) ligand extraction. A sample of MIL-53(Al) made according to the above-described batch process was loaded into the first reactor and both reactors were filled with ethanol before sealing. Ethanol is pumped (0.5 ml min⁻¹) into the first reactor and flows over the sample. The flow then goes into a second reactor which is cooled before passing through a back pressure regulator which maintains pressure of 10 MPa. The reactor heating block was heated to 260° C. (ramp rate 520° C. min⁻¹). The internal reactor temperature was 252° C. When cooling down pressure and flow was maintained until cool, the system is then slowly depressurised. The reactor was heated at 252° C. for 2 hr and 60 ml volume of ethanol pumped into the reactor.

The characterisation shown is for a MIL-53 (Al) sample made in a batch reaction however the process works equally well with the samples made according to the continuous flow process of the invention.

FIG. 7 shows the ATR-IR (Attenuated Total Reflectance Infrared) spectroscopy of as synthesised MIL-53(Al), MIL-53(Al) post scEtOH extraction, and terephthalic acid. This shows the C═O stretching peak for free terephthalic acid at 1700 cm⁻¹, the peak height and area of this peak are reduced by 90% after scEtOH extraction. These measurements were performed using Thermo Scientific Nicolet iS5 with an iD5 ATR.

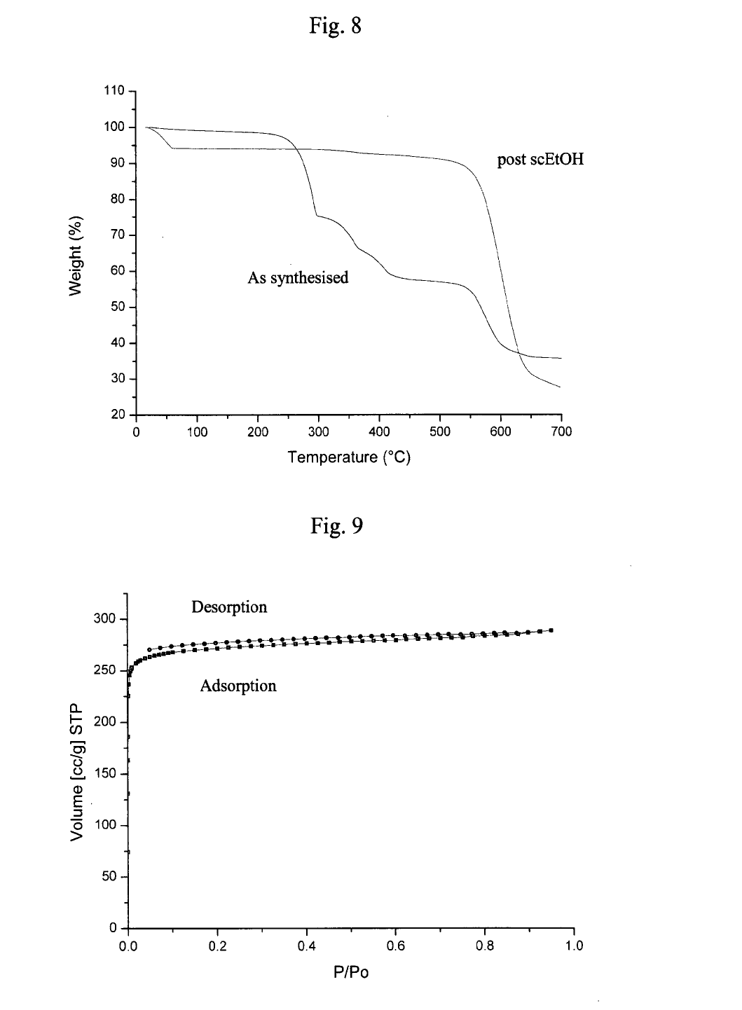

FIG. 8 show a TGA (thermogravimetric analysis) plot for both as synthesised and ligand removed by scEtOH samples. The TGA is in agreement with the ATR-IR and shows a weight drop of 2.9% between 200° C. and 500° C. for the post scEtOH sample whereas the as synthesised sample shows a drop of 41.6%. Therefore it shows a large reduction in weight loss in the region for sublimation of terephthalic acid. These measurements were performed using Perkin Elmer Pryis 1 TGA (model no. R1R151 TGA, software Ver. 11.0.0.0449). A heating rate of 5° C. min⁻¹ was used from room temperature up to 700° C.

Therefore these characterisation methods agree that 90-93% of the terephthalic acid is removed from the pores.

FIG. 9 shows a Type I N₂ isotherm at 77 K for new MIL-53 (Al) sample treated with scEtOH. Below is the N₂ isotherm measured used to measure the gas uptake and surface area. The maximum N₂ uptake is 288.7 cm³ g⁻¹ and a BET surface area of 1097 m² g⁻¹.

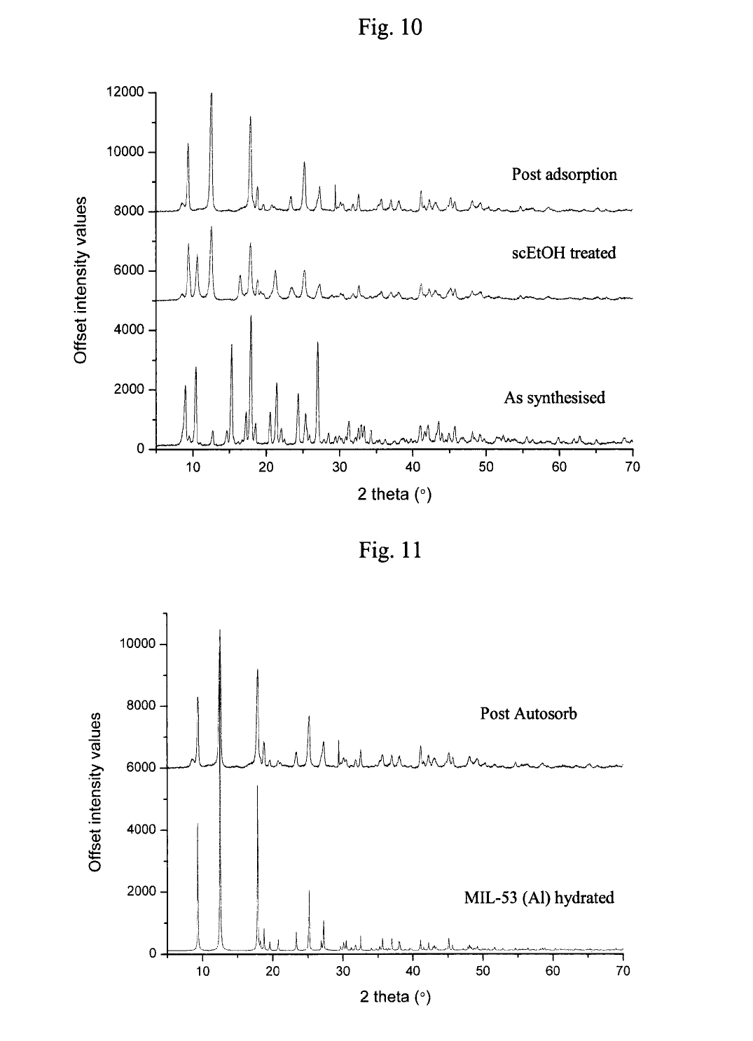

FIG. 10 shows a PXRD of the sample before and after scEtOH washing, and after N₂ adsorption. These measurements were performed using Pananalytical X'Pert Pro diffractometer operated at 160 W (40 kV, 40 mA) for Cu Kα1 (λ=1.5406 Å). The phase is different for each stage. PXRD shows the phase for each stage, the as synthesised sample is a mixture of MIL-53 (Al) hydrated, dehydrated and containing terephthalic acid in the pores phases. The phase after scEtOH washing is new and contains ethanol in the pores, and the phase after the gas adsorption is the hydrated phase (moisture adsorbed from air after N₂ adsorption).

FIG. 11 is a PXRD that shows that after 24 hrs of storage in air after gas adsorption, the phase is almost completely hydrated. PXRD run immediately after the gas sorption shows a mixture of hydrated and dehydrated phases. This characterisation shows the metal-organic framework is not damaged by the extraction process and maintains its porosity.

Thus, the present invention provides a method for extracting unreacted ligand from a metal-organic framework using a supercritical fluid.

It will be appreciated by those skilled in the art that the foregoing is a description of a preferred embodiment of the present invention and that variations in design and construction may be made to the preferred embodiment without departing from the scope of the invention as defined by the appended claims.

1. A continuous flow process for synthesising a metal-organic framework comprising the steps of:
a. providing a ligand and a metal salt which are suitable for forming a metal-organic framework,
b. mixing the ligand, metal salt and, optionally, other reagents with a solvent to form a mixture, and
c. providing the mixture at a temperature sufficient to cause the ligand and the metal salt to react to form a metal-organic framework.

2. The continuous flow process according to claim 1 wherein the solvent is preheated to a temperature sufficient to cause the ligand to react with the metal salt to form a metal-organic framework.

3. The continuous flow process according to claim 2 wherein the preheated solvent is a supercritical fluid or near critical fluid.

4. The continuous flow process according to claim 2 wherein the preheated solvent is at a temperature of at least about 200° C.

5. The continuous flow process according to claim 2 wherein the preheated solvent is at a pressure of at least about 1.55 MPa.

6. The continuous flow process according to claim 1 wherein the mixture is at a temperature of at least about 150° C., preferably at least 200° C.

7. The continuous flow process according to claim 1 wherein mixture comprises supercritical or near critical solvent.

8. The continuous flow process according to claim 1 wherein the solvent is water and/or ethanol.

9. A method for the treatment of a metal-organic framework to increase its available internal surface area and/or porosity comprising the steps of:
a. providing a metal-organic framework formed by reacting a ligand and a metal salt;
b. introducing a supercritical fluid or near critical fluid into the metal-organic framework; and
c. removing the supercritical fluid or near critical fluid;
characterised in that unreacted ligand is soluble in the supercritical fluid or near critical fluid.

10. The method according to claim 9 wherein the supercritical fluid or near critical fluid is ethanol.

11. The method according to claim 9 wherein the supercritical fluid or near critical fluid is used in combination with a co-solvent.

12. The method according to claim 11 wherein the co-solvent is a supercritical fluid or near fluid.

13. The method according to claim 12 wherein the co-solvent is supercritical carbon dioxide.

14. The method according to claim 9 characterised in that the metal-organic framework is made according to a process according to claim 1.

15. A method for synthesising a metal-organic framework using recycled unreacted ligand comprising the steps of:
a. providing a first metal-organic framework firmed by reacting a ligand and a metal salt;
b. extracting any unreacted ligand present first metal-organic framework; and
c. using said extracted unreacted ligand to synthesise a second metal-organic framework.

16. The method according to claim 15 wherein the first and/or second organic frameworks are synthesised according to the continuous flow process according to claim 1.

17. The method of claim 15 wherein the unreacted ligand is extracted according to the method of claim 9.

18. (canceled)

19. (canceled)

20. (canceled)

21. (canceled)

22. (canceled)

23. (canceled)

24. (canceled)

25. (canceled)

26. (canceled)
